(19) United States
(12) Patent Application Publication
(10) Pub. No.: US 2005/0166275 A1
(43) Pub. Date: Jul. 28, 2005

(54) Transgenic animal model for chronic pancreatitis

(75) Inventors: Herbert Archer JR., Westbury, NY (US); Dafna Bar-Sagi, Stony Brook, NY (US)

Correspondence Address: MEDLEN & CARROLL, LLP, Suite 350, 101 Howard Street, San Francisco, CA, 94105, US

(73) Assignee: The Research Foundation of State University of New York, Albany, NY (US)

(21) Appl. No.: 10/888,462

(22) Filed: Jul. 9, 2004

Related U.S. Application Data
(60) Provisional application No. 60/486,143, filed on Jul. 10, 2003.

(51) Int. Cl. A01K 67/027

(52) U.S. Cl. 800/18

(57) ABSTRACT

The present invention relates to transgenic animal models of chronic pancreatitis. The present invention also provides methods for generating animal models by introducing mutated trypsinogen genes into the germline of animals and screening methods for identifying biologically active compound.

This application claims priority to provisional patent application Ser. No. 60/286,143, filed Jul. 10, 2003; which is herein incorporated by reference in its entirety.

This invention was made with government support under Grant No. NIH/NCI R37 CA55360 awarded by the National Institutes of Health. The Government has certain rights in the invention.

### FIELD OF THE INVENTION

The present invention relates to transgenic animal models of chronic pancreatitis. The present invention also provides methods for generating animal models by introducing mutated trypsinogen genes into the germline of animals and screening methods for identifying biologically active compounds.

### BACKGROUND

Chronic pancreatitis is an inflammatory disease that causes structural and functional damage to functioning glandular tissue pancreas. This damage results in exocrine and endocrine defects. In particular, the lack of pancreatic enzymes interferes with the ability to properly digest fat. The production of insulin is also affected, which can lead to diabetes. The symptoms of chronic pancreatitis include attacks of abdominal pain, nausea, vomiting, weight loss and fatty stools.

About 70-80 percent of chronic pancreatitis cases occur in patients with a long term history of alcohol abuse. A smaller subset of chronic pancreatitis cases are hereditary in nature. The majority of these patients develop symptoms before the age of 20, and often before the age of five.

Recent genetic discoveries have identified mutations in the trypsinogen gene and protein that appear to be responsible for hereditary chronic pancreatitis. See, e.g., Whitcomb et al., Nature Genet. 14: 141-51 (1996); Gorry et al., Gastroenterology 113: 1063-8 (1997); Teich et al., Human Mutation 12: 39-43 (1998); Creighton et al., Br. J. Surg. 87: 170-5 (2000); Monaghan et al., Am. J. Med. Genet. 94: 120-4 (2000); Nishimori et al., Gut 44: 259-63 (1999); Truninger et al., Pancreas 22: 18-23 (2001); and Truninger et al., Swiss Med. Wkly. 131: 565-74 (2001). Three mutations have been identified in families exhibiting hereditary pancreatitis. A G to A transition in exon 3 of the PRSS1 gene was identified in affected individual in 5 hereditary pancreatitis families. This mutation results in an arginine (R) to histidine (H) substitution at amino acid residue 122 of trypsinogen according to the nomenclature system for human gene mutations. A A to T transversion in exon 2 of PRSS1 was identified in two hereditary pancreatitis families that lacked the R122H mutation. This mutation results an asparagine (N) to isoleucine (I) amino acid substitution at residue 29 (N29I). The third identified mutation is a C to T transition in exon 2, resulting in an alanine to valine substitution (A16V).

Currently, there are a lack of effective preventative and therapeutic strategies for chronic pancreatitis. One reason such strategies have not been developed is because of the lack of a useful animal model for the disease state. One group attempted to produce a transgenic animal model of chronic pancreatitis by introducing a human gene having the R122H mutation into a mouse. Ulrich et al., Hereditary pancreatitis: Epidemiology, molecules, mutations, and models. J. Lab. Clin. Med. 136(4): 260-274 (2000). The authors specifically stated that the human gene must be used. However, according to the authors, the transgenic mice failed to demonstrate abnormalities in pancreatic morphology or histology after 12 months on a high carbohydrate diet.

Accordingly, what is needed are better animal models of chronic pancreatitis.

### SUMMARY OF THE INVENTION

The present invention relates to transgenic animal models of chronic pancreatitis. The present invention also provides methods for generating animal models by introducing mutated trypsinogen genes into the germline of animals and screening methods for identifying biologically active compounds.

Accordingly, in some embodiments the present invention provides a transgenic animal whose genome comprises a heterologous mutant trypsinogen gene, wherein said animal exhibits a phenotype selected from the group consisting of inflammatory destruction of exocrine pancreas, pre-neoplastic changes to pancreatic duct cells, periductal chronic inflammation, up-regulation of MMP-7, elevated fasting blood glucose levels, and high peaks in blood glucose after glucose administration and combinations thereof. The present invention is not limited to transgenic animals containing any particular mutant trypsinogen gene. Indeed, transgenic animals comprising a variety of mutant trypsinogen genes are contemplated. In some embodiments, the heterologous mutant trypsinogen gene encodes a trypsinogen protein having an altered autolysis site. In other embodiments, the heterologous mutant trypsinogen gene encodes a trypsinogen protein having a substitution mutation at a position corresponding to R122 of human trypsinogen. In still other embodiments, the heterologous gene comprises an arginine to histidine mutation at a position corresponding to amino acid 122 of human trypsinogen. In further embodiments, the heterologous mutant trypsinogen gene encodes a trypsinogen protein having a substitution mutation at a position selected from the group consisting of positions corresponding to R122, A16 and N29 of human trypsinogen and combinations thereof. The present invention is not limited to any particular transgenic animal. Indeed, a variety of transgenic animals are contemplated, including, but not limited to rodents such mouse, rats, rabbits, and hamsters and non-human primates.

In some embodiments, the present invention provides methods of making a transgenic animal comprising the steps of: a) introducing into an oocyte or embryonal cell of a non-human animal a polynucleotide sequence derived from said non-human animal that encodes a mutant trypsinogen protein, under conditions such that an embryonal target cell is produced; b) transplanting said embryonal target cell, into a recipient female parent under conditions such that at least one offspring is produced; and c) identifying said at least one offspring of said recipient female parent containing said polynucleotide sequence encoding said mutant trypsinogen protein, wherein said animal exhibits a phenotype selected from the group consisting of inflammatory destruction of exocrine pancreas, pre-neoplastic changes to pancreatic duct cells, periductal chronic inflammation, up-regulation of MMP-7, elevated fasting blood glucose levels, and high peaks in blood glucose after glucose administration and combinations thereof. The present invention is not limited to transgenic animals containing any particular mutant trypsinogen gene. Indeed, transgenic animals comprising a variety of mutant trypsinogen genes are contemplated. In some embodiments, the heterologous mutant trypsinogen gene encodes a trypsinogen protein having an altered autolysis site. In other embodiments, the heterologous mutant trypsinogen gene encodes a trypsinogen protein having a substitution mutation at a position corresponding to R122 of human trypsinogen. In still other embodiments, the heterologous gene comprises an arginine to histidine mutation at a position corresponding to amino acid 122 of human trypsinogen. In further embodiments, the heterologous mutant trypsinogen gene encodes a trypsinogen protein having a substitution mutation at a position selected from the group consisting of positions corresponding to R122, A16 and N29 of human trypsinogen and combinations thereof. The present invention is not limited to any particular transgenic animal. Indeed, a variety of transgenic animals are contemplated, including, but not limited to rodents such mouse, rats, rabbits, and hamsters and non-human primates.

In still other embodiments, the present invention provides methods of identifying compounds, comprising: a) providing at least one test compound and an animal whose genome comprises a heterologous mutant trypsinogen gene, wherein said animal exhibits a phenotype selected from the group consisting of inflammatory destruction of exocrine pancreas, pre-neoplastic changes to pancreatic duct cells, periductal chronic inflammation, up-regulation of MMP-7, elevated fasting blood glucose levels, and high peaks in blood glucose after glucose administration and combinations thereof; b) exposing said transgenic animal to said at least one test compound; and c) detecting a change in at least one of said phenotypes in the presence of said test compound relative to the absence of said test compound. In some embodiments, the test compound is a drug candidate. The present invention is not limited to transgenic animals containing any particular mutant trypsinogen gene. Indeed, transgenic animals comprising a variety of mutant trypsinogen genes are contemplated. In some embodiments, the heterologous mutant trypsinogen gene encodes a trypsinogen protein having an altered autolysis site. In other embodiments, the heterologous mutant trypsinogen gene encodes a trypsinogen protein having a substitution mutation at a position corresponding to R122 of human trypsinogen. In still other embodiments, the heterologous gene comprises an arginine to histidine mutation at a position corresponding to amino acid 122 of human trypsinogen. In further embodiments, the heterologous mutant trypsinogen gene encodes a trypsinogen protein having a substitution mutation at a position selected from the group consisting of positions corresponding to R122, A16 and N29 of human trypsinogen and combinations thereof. The present invention is not limited to any particular transgenic animal. Indeed, a variety of transgenic animals are contemplated, including, but not limited to rodents such mouse, rats, rabbits, and hamsters and non-human primates.

In still other embodiments, the present invention provides an isolated and purified nucleic acid comprising a sequence encoding a protein encoded by SEQ ID NO: 3 or 4. In some embodiments, the sequence is operably linked to a heterologous promoter. In other embodiments, the sequence is contained within a vector. In other embodiments, the vector is within a host cell. In some embodiments, the present invention provides isolated and purified nucleic acid sequence encoding a protein that is at least 98% identical to SEQ ID NO: 3 or 4.

In some embodiments, the present invention provides mouse trypsinogen nucleic acid sequences selected from the group consisting of SEQ ID NO:2 and sequences that hybridize to SEQ ID NO:2 under highly stringent conditions, wherein said hybridizing sequences encode a protein having a mutation at a position corresponding to R122 of human trypsinogen and proteins encoded by such sequences.

### DESCRIPTION OF THE DRAWINGS

FIG. 1 shows the sequences of SEQ ID NOs: 1 (wild-type mouse pancreatic trypsinogen) and 2 (mutant mouse pancreatic trypsinogen).

FIGS. 2A and B provide protein translation products (SEQ ID NOs: 3 and 4) for the mouse trypsinogen nucleic acids of SEQ ID NOs: 1 and 2, respectively. The protein translations of the remaining mouse trypsinogen isoforms, containing the unique R122H mutation, are also provided (SEQ ID NOs: 5-10).

FIG. 3 provides a schematic of the linearized transgene is shown. The transgene was linearized from its plasmid background with a Sac1/PshA1 digestion. As shown, the transgene is composed of a unique mouse trypsinogen cDNA containing the R122H mutation, as well as a Rat trypsin 5′ untranslated region (UTR), and a C-terminal Flag epitope tag. This gene fusion was placed downstream of the Elastase −500/+8 promoter fragment and upstream of, sequentially, a rabbit B-globin intron, Ires-GFP element, and SV40 polyadenylation signal.

FIG. 4 provides a graph of results of glucose tolerance testing performed in 5 transgenic and four wild type animals. The results of each group were averaged, and the mean response for each group was plotted. The transgenic animal group was found to have an elevated fasting blood glucose compared to wild type, as well as an abnormal high peak in blood glucose in some of the transgenic animals.

FIG. 5 provides the nucleic acid sequences of additional mouse trypsinogen mutants (SE ID NOs: 23-33).

FIG. 6 provides the amino acid sequences of additional mouse trypsinogen mutants (SEQ ID NOs: 34-38).

Definitions

To facilitate an understanding of the invention, a number of terms are defined below.

As used herein, the term “animal” refers to any animal (e.g., a mammal), including, but not limited to, humans, non-human primates, rodents (e.g., mice, rats, etc.), flies, and the like.

As used herein, the term “non-human animals” refers to all non-human animals including, but are not limited to, vertebrates such as rodents, non-human primates, ovines, bovines, ruminants, lagomorphs, porcines, caprines, equines, canines, felines, aves, etc.

The term “gene” refers to a nucleic acid (e.g., DNA) sequence that comprises coding sequences necessary for the production of a polypeptide or precursor. The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (including heteronuclear RNA; hnRNA and mRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

Where amino acid sequence is recited herein to refer to an amino acid sequence of a naturally occurring protein molecule, amino acid sequence and like terms, such as polypeptide or protein are not meant to limit the amino acid sequence to the complete, native amino acid sequence associated with the recited protein molecule.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified”, “mutant”, and “variant” refer to a gene or gene product that displays modifications in sequence and or functional properties (i.e., altered characteristics) when compared to the wild-type gene or gene product. It is noted that naturally occurring mutants can be isolated; these are identified by the fact that they have altered characteristics when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotides or polynucleotide, referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the terms “an oligonucleotide having a nucleotide sequence encoding a gene” and “polynucleotide having a nucleotide sequence encoding a gene,” means a nucleic acid sequence comprising the coding region of a gene or, in other words, the nucleic acid sequence that encodes a gene product. The coding region can comprise of cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids.

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5× Denhardt's reagent [50× Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The following terms are used to describe the sequence relationships between two or more polynucleotides: “reference sequence,” “sequence identity,” “percentage of sequence identity,” and “substantial identity.” A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman [Smith and Waterman, Adv. Appl. Math. 2: 482 (1981)] by the homology alignment algorithm of Needleman and Wunsch [Needleman and Wunsch, J. Mol. Biol. 48: 443 (1970)], by the search for similarity method of Pearson and Lipman [Pearson and Lipman, Proc. Natl. Acad. Sci. (U.S.A.) 85: 2444 (1988)], by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a segment of the full-length sequences of the compositions claimed in the present invention (e.g., mouse pancreatic trypsinogen).

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions that are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

As used herein, the term “mouse pancreatic trypsinogen” when used in reference to a protein or nucleic acid refers to a protein or nucleic acid encoding a protein that is a protease and shares greater than about 90% identity to the protein encoded by SEQ ID NO: 3. The term mouse pancreatic trypsinogen encompasses both proteins that are identical to wild-type mouse pancreatic trypsinogen (e.g., the trypsinogen encoded by SEQ ID NO:3) and those that are derived from wild type mouse pancreatic trypsinogen (e.g., variants such as SEQ ID NO:4).

As used herein, the term “mutant pancreatic trypsinogen” when used in reference to a protein or nucleic acid refers to a nucleic acid or protein sequence encoding a mutant trypsinogen protein that is expressed in the pancreas. For example, the mutant pancreatic trypsinogen can have a substitution mutation at the trypsin autolysis site or at the trypsin signal peptide cleavage site or both. The genes may be cDNAs or genomic DNAs. SEQ ID NO:2 provides an example of a mutant pancreatic trypsinogen gene, while SEQ ID NO:4 provides an example of a mutant pancreatic trypsinogen protein.

As used herein, the term “wild-type trypsinogen gene” refers to trypsinogen genes that encode proteins that do not have mutations at sites corresponding to amino acids 16, 29 and 122 of human trypsinogen.

As used herein, the term “altered autolysis site” refers to an amino acid substitution in a trypsinogen molecule at the site where trypsin is inactivated by cleavage.

As used herein, the term “position corresponding to . . . of human trypsinogen” refers to an amino acid position in a non-human trypsinogen molecule that functionally correspond to the stated (i.e., 16, 29, and 122) amino acid position in human trypsinogen. For example, an amino acid at a position corresponding to R122 of human trypsinogen refers to an amino acid at the position where the trypsin molecule is cleaved by autolysis.

As used herein, the term “polynucleotide sequence derived from a non-human animal” refers to a polynucleotide sequence that is initially cloned from a non-human animal and subsequently used to produce an altered gene for transfer into the non-human animal.

As used herein, the term “regulatory element” refers to a genetic element that controls some aspect of the expression of nucleic acid sequences. For example, a promoter is a regulatory element that facilitates the initiation of transcription of an operably linked coding region. Other regulatory elements include splicing signals, polyadenylation signals, termination signals, etc.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

The term “fragment” as used herein refers to a polypeptide that has an amino-terminal and/or carboxy-terminal deletion as compared to the native protein, but where the remaining amino acid sequence is identical to the corresponding positions in the amino acid sequence deduced from a full-length cDNA sequence. Fragments typically are at least 4 amino acids long, preferably at least 20 amino acids long, usually at least 50 amino acids long or longer, and span the portion of the polypeptide required for intermolecular binding of the compositions (claimed in the present invention) with its various ligands and/or substrates.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

As used herein the term “coding region” when used in reference to structural gene refers to the nucleotide sequences that encode the amino acids found in the nascent polypeptide as a result of translation of a mRNA molecule. Coding regions in eukaryotes are a composition comprising of 5′ ends with nucleotide triplets “ATG” that encode methionine and 3′ end sequences comprising of nucleotide triplets that specify stop codons (e.g., TAA, TAG, TGA).

The term “recombinant DNA molecule” as used herein refers to a DNA molecule that is comprised of segments of DNA joined together by means of molecular biological techniques.

The term “recombinant protein” or “recombinant polypeptide” as used herein refers to a protein molecule that is expressed from a recombinant DNA molecule.

The term “native protein” as used herein to indicate that a protein does not contain amino acid residues encoded by vector sequences; that is the native protein contains only those amino acids found in the protein as it occurs in nature. A native protein may be produced by recombinant means or may be isolated from a naturally occurring source.

The term “transgene” as used herein refers to a foreign, heterologous, or autologous gene that is placed into an organism by any method. The term “foreign gene” refers to any nucleic acid (e.g., gene sequence) that is introduced into the genome of an animal by experimental manipulations and may include gene sequences found in that animal so long as the introduced gene does not reside in the same location as does the naturally-occurring gene.

As used herein, the term “transgenic animal” refers to any animal containing a transgene.

As used herein, the term “vector” is used in reference to nucleic acid molecules that transfer DNA segment(s) from one cell to another. Vector can include partial genes, gene fragments, full length genes and target sequences. The term “vehicle” is sometimes used interchangeably with “vector.”

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells utilize promoters, enhancers, and termination and polyadenylation signals.

As used herein, the term host cell refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic animal.

The terms “overexpression” and “overexpressing” and grammatical equivalents, are used in reference to levels of mRNA to indicate a level of expression greater (e.g., approximately 2-fold or more higher) than that typically observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed, often referred to as “housekeeping” genes (e.g., the amount of 28S rRNA, an abundant RNA transcript present at essentially the same amount in all tissues, present in each sample can be used as a means of normalizing or standardizing the mRNA-specific signal observed on Northern blots).

The term “transfection” as used herein refers to the introduction of foreign DNA into cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign DNA into the genome of the transfected cell. The term “stable transfectant” refers to a cell that has integrated foreign DNA into its genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for up to several cell divisions. During this time the foreign DNA is subject to the regulatory controls that govern the expression of endogenous genes in the chromosomes. The term “transient transfectant” refers to cells that have taken up foreign DNA but have failed to integrate this DNA.

The term “test compound” refers to any chemical entity, pharmaceutical, drug, and the like that can be used to treat or prevent a disease, illness, sickness, or disorder of bodily function, or otherwise alter the physiological or cellular status of a sample. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

The term “sample” as used herein is used in its broadest sense. A sample suspected of containing a human chromosome or sequences associated with a human chromosome may comprise a cell, chromosomes isolated from a cell (e.g., a spread of metaphase chromosomes), genomic DNA (in solution or bound to a solid support such as for Southern blot analysis), RNA (in solution or bound to a solid support such as for Northern blot analysis), cDNA (in solution or bound to a solid support) and the like. A sample suspected of containing a protein may comprise cell lysate, a cell, a portion of a tissue, tissue lysate and the like.

### DETAILED DESCRIPTION OF THE INVENTION

The present invention relates to transgenic animal models of chronic pancreatitis. The present invention also provides methods for generating animal models by introducing mutated trypsinogen genes into the germline of animals and screening methods for identifying biologically active compounds. Thus, the present invention provides transgenic animals having somatic and germ cells comprising a heterologous, mutant trypsinogen gene. Preferred embodiments of the present invention are illustrated below using the example of a mouse model. Such animals find use in a variety of applications, including, but not limited to, those described below.

The present invention is not limited to any particular mechanism. Indeed, an understanding of the mechanism of action of the present invention is not necessary to practice the present invention. However, the primary hydrolytic site of trypsin by trypsin itself or other proteases is known to the arginine at position R122. Thus, it is likely that mutations at this site (e.g., the R122H mutation) eliminate the primary autolysis site, rendering the mutant trypsin resistant to autolysis and permanent inactivation. The functional consequences of the A16V mutation are less clear. A16 is the signal peptide cleavage site; therefore, mutations in that region may disrupt intracellular transport of pretypsinogen or cause a conformational change in the activation peptide that causes enhanced autoactivation. The N29I mutation has been found to stabilize the precursor zymogen form of trypsinogen. It is believed that this mutation renders the precursor resistant to the cleavage that normally occurs when other pools of trypsin are activated. Thus, the autodigestive process that is altered is the autodigestion of the precursor pool of trypsinogen, not the activated enzyme as with the R122H mutation. This can lead to a buildup of precursor pools, that when finally activated, can have the same autodigestive effects as the R122H mutants.

The present invention provides transgenic animals comprising a heterologous trypsinogen gene derived from the animal that encodes a mutation corresponding to amino acids 122, 16, or 29 of human trypsinogen. For example, an amino acid corresponding to position 122 in a non-human animal is the amino acid at the primary autolysis site, while an amino acid corresponding to position 16 in a non-human animal is an amino acid at the signal peptide cleavage site. The heterologous trypsinogen genes can have mutations at one or more of the following positions corresponding to the human amino position: 16, 29, and 122.

The present invention is demonstrated by the production of transgenic mice comprising a R122H mouse trypsinogen transgene. Experiments conducted during the course of development of the present invention demonstrated that R122H trypsinogen transgenic mice demonstrate the presence of a disease closely resembling the human condition of hereditary pancreatitis; characterized by the early onset of repeated bouts of acute pancreatitis that resolve into a state of chronic pancreatitis, from which the ductal epithelium is stimulated to proliferate in an uncontrolled fashion, leading to the preneoplastic changes observed histologically and neoplastic progression often seen clinically.

The following aspects of the invention are described in more detail below: I. Transgenes and Vector Constructs; II. Production of Transgenic Animals; and III. Drug Screening.

I. Transgenes and Vector Constructs

The present invention provides novel genes and vector constructs for the production of transgenic animal models of chronic pancreatitis. In some embodiments of the present invention, the genes are derived from trypsinogen genes that are native to the animal into which the gene will be introduced. For example, to construct mouse trypsinogen genes of the present invention, the native trypsinogen genes are isolated from a mouse. As another example, to construct rat trypsinogen genes of the present invention, the native trypsinogen genes are isolated from a rat. In some embodiments, the isolated native trypsinogen genes are then altered by site-specific mutagenesis (e.g., introduction of mutation via the use of PCR primers) or other appropriate technique to encode proteins having substitution mutations at one or more of the following amino acid positions: R122, N29, or A16 or the corresponding position in the isolated trypsinogen. The mutant trypsinogen gene is then cloned into an appropriate expression vector.

As described above, the present invention is not limited to the use of genes into which any particular mutation has been introduced or to genes derived from any particular species. In some embodiments, the present invention provides genes with one or more of the following mutations (or corresponding to the following mutations in the human gene): R122H, N29I, and A16V. The present invention is not limited to substitution of the amino acids at sites R122, N29 and A16 with any particular amino acid. Indeed, any of the other 19 amino acids can be substituted at these positions. In preferred embodiments, the gene is a mouse trypsinogen gene encoding a trypsinogen protein having a R122H mutation. In further preferred embodiments, the mutant mouse trypsinogen gene is encoded by the following nucleic acid sequences: SEQ ID NO: 2 and SEQ ID NOs: 23-33) or sequences which bind to the foregoing sequences under high stringency conditions and have at least one of mutations R122H, N29I and A16V or the appropriate corresponding mutation. In other embodiments, the nucleic acid sequence encodes a mutant protein selected from the group consisting of SEQ ID NOs: 4-10 and 34-38.

Additional mutant trypsinogen genes with alteration in addition to those identified above are also within the scope of the present invention. In particular, it is contemplated that it is possible to further modify the structure of a peptide having a function (e.g., mutant trypsinogen function). Such modified peptides are considered functional equivalents of peptides having an activity of a mutant trypsinogen (e.g., resistance to autolysis). A modified peptide can be produced in which the nucleotide sequence encoding the polypeptide has been altered, such as by substitution, deletion, or addition. The construct can be evaluated in order to determine whether it is a member of the genus of modified or variant trypsinogens of the present invention as defined functionally, rather than structurally. In preferred embodiments, the activity of the variant or mutant trypsinogen is evaluated by the methods described below.

Moreover, as described above, variant forms of trypsinogen are also contemplated as being equivalent to those peptides and DNA molecules that are set forth in more detail herein. For example, it is contemplated that isolated replacement of a leucine with an isoleucine or valine, an aspartate with a glutamate, a threonine with a serine, or a similar replacement of an amino acid with a structurally related amino acid (i.e., conservative mutations) will not have a major effect on the biological activity of the resulting molecule. Accordingly, some embodiments of the present invention provide variants of trypsinogen disclosed herein containing conservative replacements. Conservative replacements are those that take place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. In similar fashion, the amino acid repertoire can be grouped as (1) acidic (aspartate, glutamate); (2) basic (lysine, arginine, histidine), (3) aliphatic (glycine, alanine, valine, leucine, isoleucine, serine, threonine), with serine and threonine optionally be grouped separately as aliphatic-hydroxyl; (4) aromatic (phenylalanine, tyrosine, tryptophan); (5) amide (asparagine, glutamine); and (6) sulfur-containing (cysteine and methionine) (e.g., Stryer ed., Biochemistry, pg. 17-21, 2nd ed, WH Freeman and Co., 1981). Whether a change in the amino acid sequence of a peptide results in a functional homolog can be readily determined by assessing the ability of the variant peptide to function in a fashion similar to the wild-type protein. Peptides having more than one replacement can readily be tested in the same manner.

A variant can also include “nonconservative” changes (e.g., replacement of a glycine with a tryptophan). Analogous minor variations can also include amino acid deletions or insertions, or both. Guidance in determining which amino acid residues can be substituted, inserted, or deleted without abolishing biological activity can be found using computer programs (e.g., LASERGENE software, DNASTAR Inc., Madison, Wis.).

It will be recognized that suitable transgenes for use in the present invention can be screened by isolating trypsinogen transgenes from a particular animal species, altering the transgene by site specific mutagenesis, inserting the transgene into a vector, creating a transgenic animal expressing the transgene, and analyzing the animal for symptoms of chronic pancreatitis. As described in more detail below, methods are available for creating many different species of transgenic animals.

In some embodiments, the trypsinogen transgene is operably linked to a suitable promoter (e.g., a heterologous promoter) to form an expression vector. In some preferred embodiments of the present invention, mammalian expression vectors comprise an origin of replication, a suitable promoter and enhancer, and also any necessary ribosome binding sites, polyadenylation sites, splice donor and acceptor sites, transcriptional termination sequences, and 5′ flanking non-transcribed sequences. In other embodiments, DNA sequences derived from the SV40 splice, and polyadenylation sites may be used to provide the required non-transcribed genetic elements. The promoter may be constitutive, but is preferably tissue specific. In some preferred embodiments, the promoter is an elastase promoter. In some preferred embodiments, the transgene coding sequence is provided in operable association with an intron (e.g., the beta-globin intron) and an IRES sequence. The IRES sequence allows co-expression of selectable or non-selectable marker gene (e.g., GFP).

II. Production of Transgenic Animals

In preferred embodiments, the transgenic animal of the present invention display an altered phenotype as compared to wild-type animals. In preferred embodiments, the transgenic animals display one or more of the following phenotypes: inflammatory destruction of exocrine pancreas, pre-neoplastic changes to pancreatic duct cells, periductal chronic inflammation, up-regulation of MMP-7, elevated fasting blood glucose levels, and high peaks in blood glucose after glucose administration.

The present invention is not limited to a particular transgenic animal. A variety of human and non-human animals are contemplated. For example, in some embodiments, rodents (e.g., mice or rats) or primates are provided as animal models of chronic pancreatitis.

The transgenic animals can be generated via a variety of methods. In some embodiments, embryonal cells (also referred to as embryonal target cells) at various developmental stages are used to introduce transgenes for the production of transgenic animals. Different methods are used depending on the stage of development of the embryonal cell. The zygote is the best target for micro-injection. In the mouse, the male pronucleus reaches the size of approximately 20 micrometers in diameter, which allows reproducible injection of 1-2 picoliters (pl) of DNA solution. The use of zygotes as a target for gene transfer has a major advantage in that in most cases the injected DNA will be incorporated into the host genome before the first cleavage (Brinster et al., Proc. Natl. Acad. Sci. USA 82: 4438-4442 [1985]). As a consequence, all cells of the transgenic non-human animal will carry the incorporated transgene. This will in general also be reflected in the efficient transmission of the transgene to offspring of the founder since 50% of the germ cells will harbor the transgene. U.S. Pat. No. 4,873,191 describes a method for the micro-injection of zygotes; the disclosure of this patent is incorporated herein in its entirety.

In other embodiments, retroviral infection is used to introduce transgenes into a non-human animal. In some embodiments, the retroviral vector is utilized to transfect oocytes by injecting the retroviral vector into the perivitelline space of the oocyte (U.S. Pat. No. 6,080,912, incorporated herein by reference). In other embodiments, the developing non-human embryo can be cultured in vitro to the blastocyst stage. During this time, the blastomeres can be targets for retroviral infection (Janenich, Proc. Natl. Acad. Sci. USA 73: 1260 [1976]). Efficient infection of the blastomeres is obtained by enzymatic treatment to remove the zona pellucida (Hogan et al., in Manipulating the Mouse Embryo, Cold Spring Harbor Laboratory Press, Cold Spring Harbor, N.Y. [1986]). The viral vector system used to introduce the transgene is typically a replication-defective retrovirus carrying the transgene (Jahner et al., Proc. Natl. Acad. Sci. USA 82: 6927 [1985]). Transfection is easily and efficiently obtained by culturing the blastomeres on a monolayer of virus-producing cells (Van der Putten, supra; Stewart, et al., EMBO J., 6: 383 [1987]). Alternatively, infection can be performed at a later stage. Virus or virus-producing cells can be injected into the blastocoele (Jahner et al., Nature 298: 623 [1982]). Most of the founders will be mosaic for the transgene since incorporation occurs only in a subset of cells that form the transgenic animal. Further, the founder may contain various retroviral insertions of the transgene at different positions in the genome that generally will segregate in the offspring. In addition, it is also possible to introduce transgenes into the germline, albeit with low efficiency, by intrauterine retroviral infection of the midgestation embryo (Jahner et al., supra [1982]). Additional means of using retroviruses or retroviral vectors to create transgenic animals known to the art involves the micro-injection of retroviral particles or mitomycin C-treated cells producing retrovirus into the perivitelline space of fertilized eggs or early embryos (PCT International Application WO 90/08832 [1990], and Haskell and Bowen, Mol. Reprod. Dev., 40: 386 [1995]).

In other embodiments, the transgene is introduced into embryonic stem cells and the transfected stem cells are utilized to form an embryo. ES cells are obtained by culturing pre-implantation embryos in vitro under appropriate conditions (Evans et al., Nature 292: 154 [1981]; Bradley et al., Nature 309: 255 [1984]; Gossler et al., Proc. Acad. Sci. USA 83: 9065 [1986]; and Robertson et al., Nature 322: 445 [1986]). Transgenes can be efficiently introduced into the ES cells by DNA transfection by a variety of methods known to the art including calcium phosphate co-precipitation, protoplast or spheroplast fusion, lipofection and DEAE-dextran-mediated transfection. Transgenes may also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal (for review, See, Jaenisch, Science 240: 1468 [1988]). Prior to the introduction of transfected ES cells into the blastocoel, the transfected ES cells may be subjected to various selection protocols to enrich for ES cells which have integrated the transgene assuming that the transgene provides a means for such selection. Alternatively, the polymerase chain reaction may be used to screen for ES cells that have integrated the transgene. This technique obviates the need for growth of the transfected ES cells under appropriate selective conditions prior to transfer into the blastocoel.

In still other embodiments, the transgene is introduced into somatic cells, and the somatic cell (either quiescent or proliferating) is fused to an enucleated oocyte via nuclear transfer. Thus, the transgene is introduced into the enucleated oocyte. These procedures are described in detail in U.S. Pat. Nos. 5,945,577; 6,147,276; and 6,235,969, all of which are incorporated herein by reference.

In still other embodiments, homologous recombination is utilized to knock-out the native trypsinogen gene. Methods for homologous recombination are described in U.S. Pat. No. 5,614,396, incorporated herein by reference.

III. Uses of Transgenic Animals

The transgenic animals of the present invention find use in a variety of applications, including, but not limited to, identification of biochemical pathways implicated in chronic pancreatitis and in drug screening applications. For example, in some embodiments, test compounds (e.g., compounds suspected of alleviating the symptoms of chronic pancreatitis) and control compounds (e.g., a placebo) are administered to the transgenic animals and the control animals. The effects of the test and control compounds on disease symptoms are then assessed. For example, in some embodiments, drug candidates are tested for the ability to alter pancreas inflammation, preneoplastic changes, periductal inflammation, regulation of MMP-7, fasting blood glucose levels, and peaks in blood glucose levels after glucose administration.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994])); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12: 145).

Examples of methods for the synthesis of molecular libraries can be found in the art, for example in: DeWitt et al., Proc. Natl. Acad. Sci. U.S.A. 90: 6909 [1993]; Erb et al., Proc. Nat'l. Acad. Sci. USA 91: 11422 [1994]; Zuckermann et al., J. Med. Chem. 37: 2678 [1994]; Cho et al., Science 261: 1303 [1993]; Carrell et al., Angew. Chem. Int. Ed. Engl. 33.2059 [1994]; Carell et al., Angew. Chem. Int. Ed. Engl. 33: 2061 [1994]; and Gallop et al., J. Med. Chem. 37: 1233 [1994].

Where the screening assay is a binding assay, one or more of the molecules may be joined to a label, where the label can directly or indirectly provide a detectable signal. Various labels include radioisotopes, fluorescent compounds, chemiluminescent compounds, enzymes, specific binding molecules, particles, e.g. magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components is added in any order that provides for the requisite binding. Incubations are performed at any suitable temperature, typically between 4 and 40° C. Incubation periods are selected for optimum activity, but may also be optimized to facilitate rapid high-throughput screening.

The present invention further provides agents identified by the above-described screening assays. Accordingly, it is within the scope of this invention to further use an agent identified as described herein in an appropriate animal model (such as those described herein) to determine the efficacy, toxicity, side effects, or mechanism of action, of treatment with such an agent. Furthermore, agents identified by the above-described screening assays can be used for treatment of chronic and hereditary pancreatitis and related diseases.

Experimental

The following examples are provided in order to demonstrate and further illustrate certain preferred embodiments and aspects of the present invention and are not to be construed as limiting the scope thereof.

### EXAMPLE 1

### Isolation of Mouse Pancreatic Trypsinogen

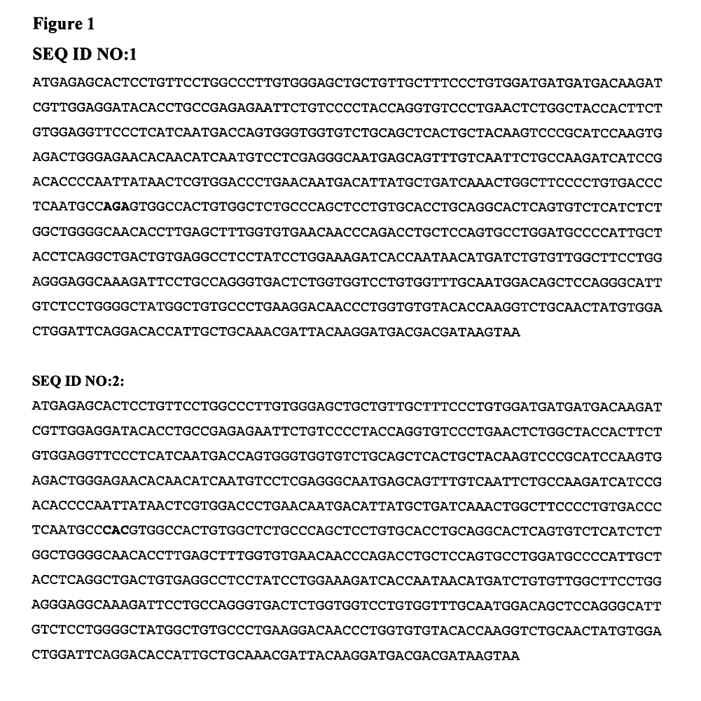

To ensure the use of an expressed isoform of mouse pancreatic trypsinogen in the generation of transgenic mice, wild-type mouse pancreatic trypsinogen was first isolated from mouse pancreas by RT-PCR. Mouse pancreatic trypsinogen was chosen for use in this model system in order to minimize the possibility of generating an immune rejection within the transgenic mouse, as well as minimizing the possibility that species differences in leader sequences might alter the localization of the chosen trypsinogen. Fresh mouse pancreas was flash frozen and ground by mortar and pestle in the presence of RLT lysis buffer (Qiagen) containing β-mercaptoethanol. Total mouse pancreas RNA was obtained by use of the RNeasy kit (Qiagen), and was shown to be intact. This RNA population was reverse transcribed using oligodT as a primer for SuperScript II Reverse Transcriptase (Invitrogen). The first strand cDNA library thus generated was used as the template for PCR using primers specific to the 5′ and 3′ ends of mouse pancreatic trypsinogen. Specifically, the primers utilized to amplify this cDNA were: 5′ CGCGGATCCATGAGAGCACTCCTGTTCCT (SEQ ID NO:11) and 3′ CGGGGTACCTTAGTTTGCAGCAATGGTG (SEQ ID NO:12). PCR was performed in duplicate and a single product was observed at the expected length of 741 bp. The sequence of this mouse trypsinogen cDNA, following the introduction of an R122H mutation via PCR mutagenesis, is shown in FIG. 1. This sequence begins at the start site of translation, and ends with the C-terminal Flag epitope tag.

This wild type PCR product was inserted into vector containing the Elastase −500/+8 promoter as a BamHI/KpnI fragment. The Elastase Promoter −500/+8 fragment was a gift from Dr. Raymond MacDonald (University of Texas, Southwestern). PCR mutagenesis was performed to generate an R122H mutation. Specifically, the elastase promoter/wild type trypsinogen construct was used as template in a first round reaction in order to introduce the Arginine to Histidine mutation. The primers used were:

(SEQ ID NO: 13)

| | |
|---|---|
| 5′ Elastase | CGCGAGCTCAAGCTTATCGTATGAA, |

(SEQ ID NO: 14)

| | |
|---|---|
| 3′ Mutant Trypsin | TGGCCACGTGGGCATTGAGGGTCA, |

(SEQ ID NO: 15)

| | |
|---|---|
| 5′ Mutant Trypsin | AATGCCCACGTGGCCACTGTGGC, |
| and | |

(SEQ ID NO: 16)

| | |
|---|---|
| 3′ Trypsin | CTAGCTAGCGGTACCTTAGTTTGCAGCAAT. |

The two fragments generated in the first round were annealed in a second round and used as template for the amplification of the full length mR122H trypsinogen mutant fused to the elastase promoter. For the second round, the 5′ Elastase and 3′ Trypsin primers listed above were used. Subsequently, the rat trypsin 5′ UTR with Kozak consensus site was added by PCR using the 5′ primer CGCGGATCCACCTTCTGCCACCATGAGAGCACTCCTGT (SEQ ID NO: 17), and the new mR122H containing the rat trypsin 5′ UTR/Kozak site was subcloned back into the elastase promoter containing vector via a BamH1/Kpn1 ligation. The newly generated R122H trypsinogen was PCR amplified and shuttled into the promoterless vector, pBasic, into which an Ires-GFP element had been subcloned as a BglII/NheI-XbaI fragment. Separately, a C-terminal Flag epitope tagged version of mouse R122H trypsinogen was generated in a two step reaction using, sequentially, the following 3′ primers:

(SEQ ID NO: 18)

| | |
|---|---|
| Round 1 | GTCATCCTTGTAATCGTTTGCAGCAATGGT, |

(SEQ ID NO: 19)

| | |
|---|---|
| Round 2 | CTAGCTAGCTTACTTATCGTCGTCATCCTT. |

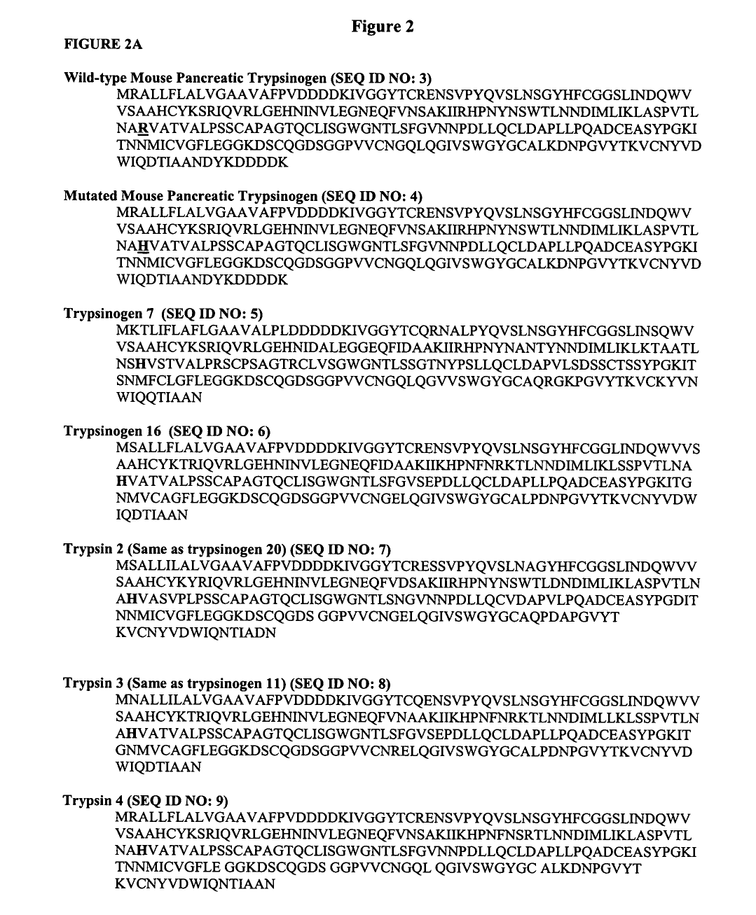

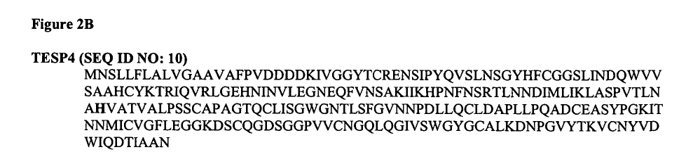

The C-terminal half of the untagged R122H was replaced with the C-terminal half of the Flag epitope tagged R122H by subcloning. Finally, the rabbit β-globin intron was obtained by PCR amplification from the pCGN expression vector, and inserted between the upstream R122H gene and the downstream Ires element via a NheI/BglII digestion. Upon generation of the final transgenic construct, the mR122H trypsinogen gene was fully sequenced and found to be a unique mouse trypsinogen gene, sharing 97% protein identity with the nearest published sequence for mouse TESP4 (see FIGS. 1 and 2).

### EXAMPLE 2

### Generation of Transgenic Mice

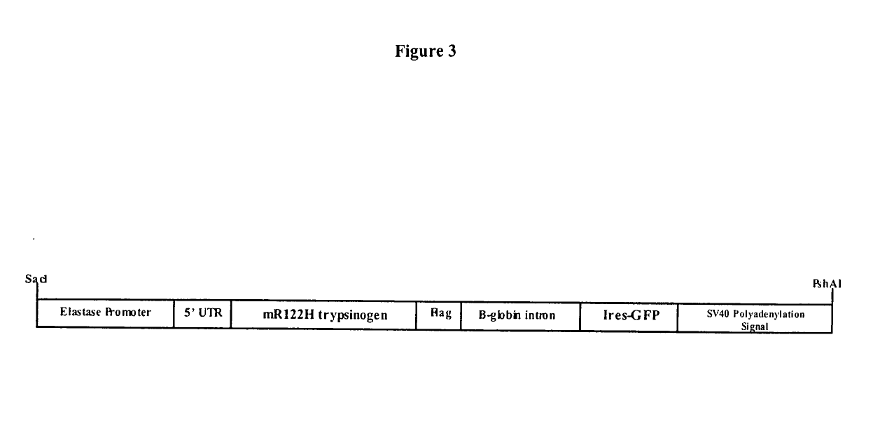

Transgenic mice bearing the mouse trypsinogen mR122H isoform were generated by the University Transgenic Mouse Facility (SUNY Stony Brook, N.Y.). Briefly, the mouse mR122H trypsinogen construct described above was linearized using the unique restriction sites Sac1 and PshA1. The 3.2 kB fragment obtained from this digestion (containing the elastase promoter, mR122H cDNA, B-globin intron, Ires-GFP, and the SV40 polyadenylation signal), was injected into the fertilized pronuclei of C57/B16 mice (FIG. 3). Mice were maintained in accordance with Institutional Animal Care and Use Committee protocols, and were bred after the age of 6 weeks. A total of 27 animals were screened for transgene incorporation using a nested PCR screen directed against the GFP portion of the transgene. Specifically, the primers used for this screen were:

(SEQ ID NO: 20)

| | |
|---|---|
| Round 1 | 5′ ATGGTGAGCAAGGGCGAGGAGCTG, |

(SEQ ID NO: 21)

| | |
|---|---|
| Round 2 | 5′ CAGCTCGTCCATGCCGAGAGTGAT, |
| and | |

(SEQ ID NO: 22)

| | |
|---|---|
| Round 1 & 2 | 3′ GCCATGCCCGAAGGCTACGTCCAG. |

Seven of the screened animals were identified as potential founders and appropriate breedings were established. F1 generations have successfully been generated from 4 of the potential founders, and each of these litters was subsequently screened for transgene incorporation. Mice from each of these litters have been sacrificed at varying time points beginning with 3 weeks of age.

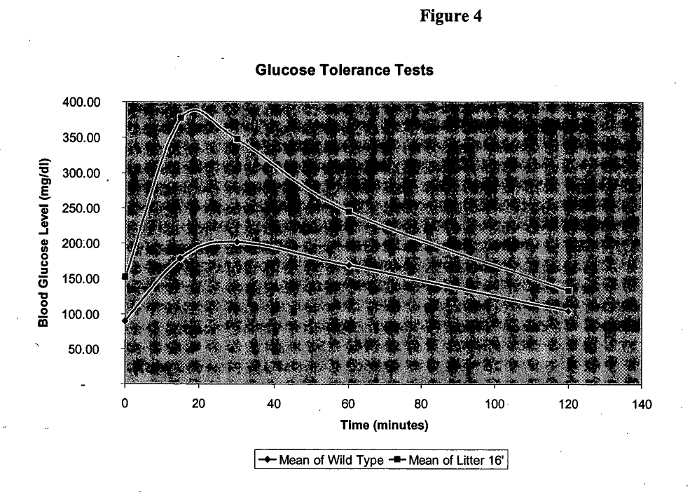

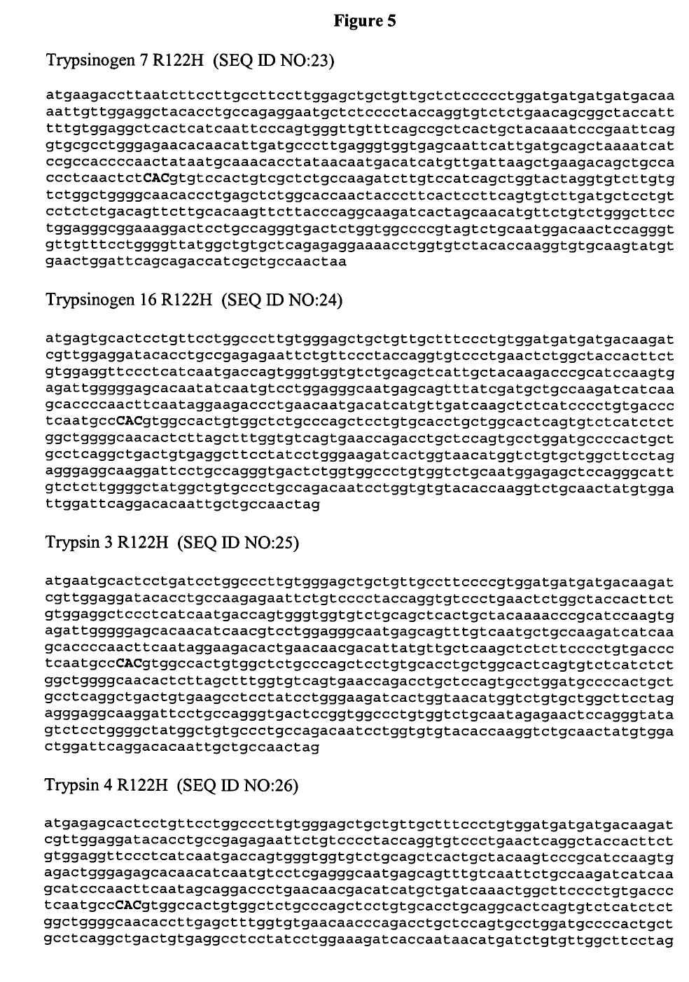

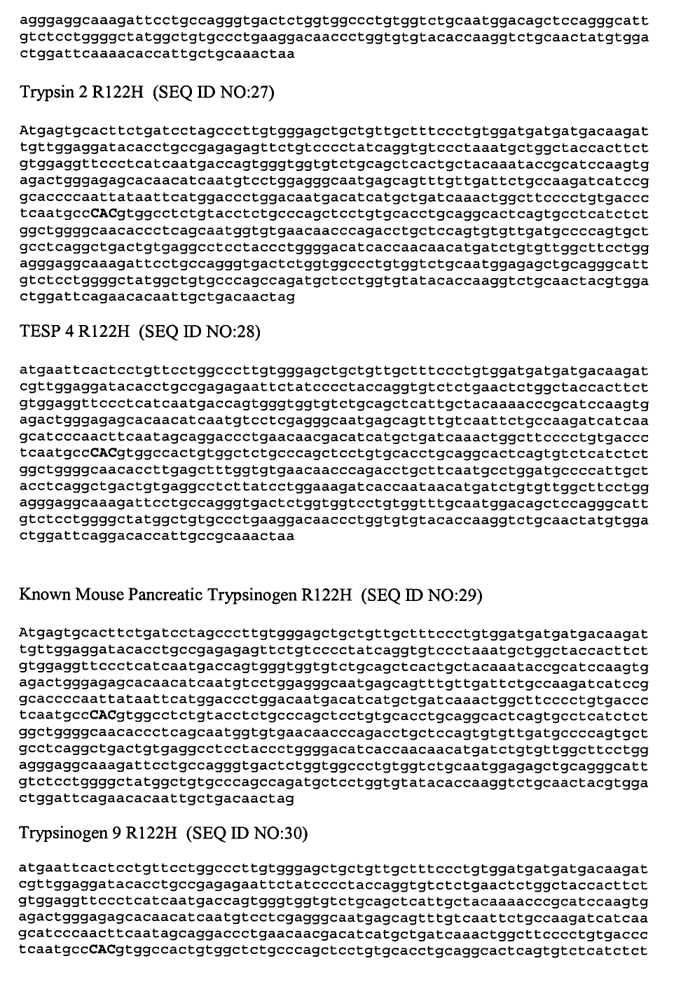

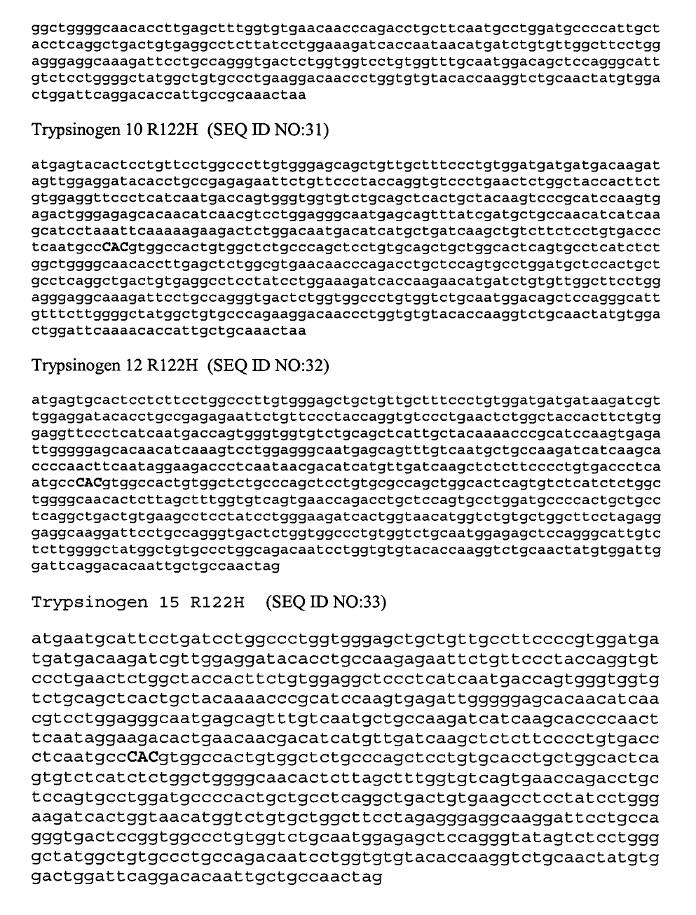

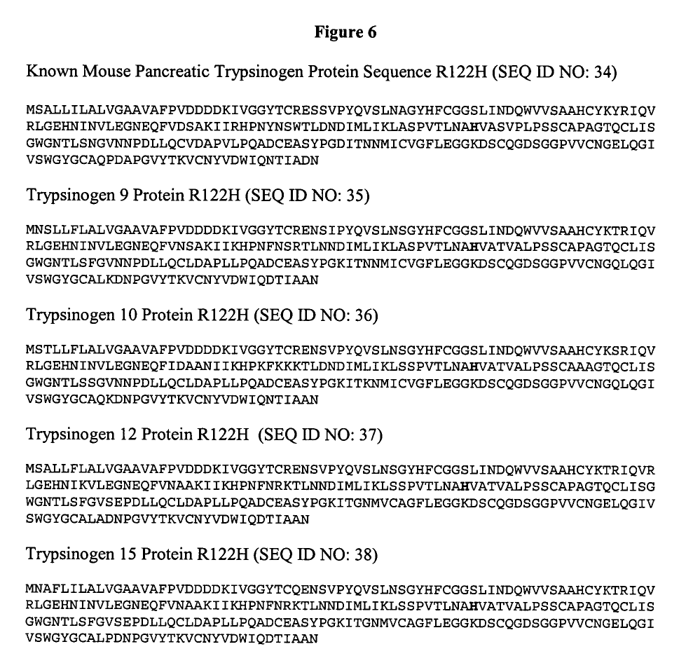

The expression of the mR122H transgene was confirmed in the animals by Western Blotting for the Flag epitope tag located on the C-terminus of mR122H trypsinogen. Briefly, the pancreatic tails of both wild-type and transgenic mice were homogenized using a Dounce homogenizer in the presence of a sucrose buffer containing protease inhibitors. Protein concentrations within the lysates were determined spectrophotometrically, and 10 and 50 μg of transgene and wild type pancreas protein was loaded on a 12.5% SDS-PAGE gel. The gel was transferred to nitrocellulose, and blotted with antibody against the Flag epitope tag. The results show the presence of two protein bands within the transgenic pancreas not found within the wild type pancreas, corresponding to the molecular weights of both trypsinogen and the activated trypsin enzyme (FIG. 4).

### EXAMPLE 3

### Histological Evaluation

Transgenic animals were sacrificed and analyzed for pancreatic morphology at varying time points after weaning. Upon sacrifice, the head of the pancreas was fixed and used for paraffin embedded sections, the body was snap frozen for frozen sections, and the tail was snap frozen for use in protein and mRNA analysis. For immunohistochemical staining, paraffin embedded pancreata were sectioned to 5 μm thickness, deparrafinized with two changes of xylene, and rehydrated through a graded alcohol series. Endogenous peroxidase activity was quenched with a 3% H₂O₂ solution, and antigenicity was recovered by boiling the sections in a 0.01M citrate buffer (pH 6.0). The sections were blocked with serum from the species in which the secondary antibody was raised, and primary antibodies were then added in the following dilutions: anti-Ki67 1:500 (Novocastra), anti-CD45 1:50 (BD Biosciences), anti MMP-7 1:1000 (gift from Dr. Howard Crawford), anti-Insulin 1:100 (Linco Research). The sections were washed with PBS or TBS after incubation with the primary antibody, and the appropriate biotinylated secondary antibody was added. Following secondary antibody incubation, the VectaStain Elite ABC Reagent (Vector Laboratories) was applied following the manufacturers instructions, and the subsequent antibody/enzyme conjugate was developed with either DAB (brown) or DAB+Nickel (gray/black). All sections were counterstained for 30 seconds with Hematoxylin and blued in dilute ammonia water. The sections were then dehydrated to 100% ethanol in a graded alcohol series, washed twice in xylene and mounted in Permount (Fisher). Hematoxylin and eosin staining was performed separately by the University Histology Services (SUNY Stony Brook, N.Y.).

Mice sacrificed as early as 7 weeks after birth revealed both inflammatory destruction of the exocrine pancreas, and the development of preneoplastic changes within the duct cells of the pancreas, representing the spectrum of human disease progression. Specifically, duct lesions have been found within the transgenic mice to include regions of proliferating, mucinous, ducts with a columnar morphology, and scattered nuclear atypia, indicative of precursor, Pancreatic Neoplasia In Situ (PanIn) lesions. Immunohistochemical analyses of the transgenic mice have indicated a conserved pattern of periductal chronic inflammation, a feature pathognomonic of the human condition. Staining for the proliferative marker, Ki-67 has revealed increased proliferation both in the ducts surrounded by inflammation as well as in the preneoplastic duct epithelium, indicating a direct role for inflammation in driving the hyperproliferation associated with tumorgenesis. In addition, routine hematoxylin and eosin staining has also revealed focal areas of acute inflammation in the transgenic mice, indicating a disease course closely mimicking the human condition, in which repeated bouts of acute inflammation resolve into a chronic inflammatory state, ultimately increasing the risk of cancer progression.

In addition to the morphological analysis, biomarkers of the human disease process have confirmed a disease state in these mice that closely recapitulates the human condition. Specifically, immunohistochemical staining for the matrix metalloproteinase, MMP-7, has revealed an upregulation of this enzyme within abnormal duct cells of our mice (data not shown). In the human disease state of chronic pancreatitis, MMP-7 has been shown to be progressively upregulated during the transition from normal duct epithelium to duct epithelium with a preneoplastic phenotype.

### EXAMPLE 4

### Glucose Tolerance Testing

Glucose tolerance testing has also been utilized to detect the presence of disease in living mice. It is known that both chronic pancreatitis and pancreatic adenocarcinoma often result in the onset of diabetes in human patients. To study whether this feature is also present in the transgenic animals, glucose tolerance testing was performed after a 16 hour fast.

Animals scheduled for glucose tolerance testing were fasted for 16 hours prior to testing. Animals were anesthetized with isoflurane, and their tails were transected to obtain tail vein blood. An initial blood glucose measurement was taken at this time=0 using a MediSense Precision QID glucose monitor. Immediately following glucose measurement, the animals were injected intraperitoneally with a 2 g/kg solution of glucose, and the animals were removed from anesthesia. Glucose measurements were then taken from the tail vein at 15, 30, 60, and 120 minutes.

Five transgenic and four wild type control animals were injected intraperitoneally with a 2 g/kg bolus of glucose, and blood glucose was measured at time 0, 15, 30, 60, and 120 minutes. The results shown in FIG. 4 reveal an appropriate peripheral response to glucose injection, but a significantly elevated fasting blood glucose level in all of the transgenic animals and an abnormally high peak in blood glucose, indicative of an early stage of diabetes. Interestingly, immunohistochemical staining for insulin in the islets of our transgenic animals has revealed abnormal islet architecture, with the insulin producing B-cells mislocalized to the periphery of the islets, indicating a general malfunction of the islets in the animals (data not shown).

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

1. A transgenic animal whose genome comprises a heterologous mutant trypsinogen gene, wherein said animal exhibits a phenotype selected from the group consisting of inflammatory destruction of exocrine pancreas, pre-neoplastic changes to pancreatic duct cells, periductal chronic inflammation, up-regulation of MMP-7, elevated fasting blood glucose levels, and high peaks in blood glucose after glucose administration and combinations thereof.

2. The transgenic animal of claim 1, wherein said heterologous mutant trypsinogen gene encodes a trypsinogen protein having an altered autolysis site.

3. The transgenic animal of claim 1, wherein said heterologous mutant trypsinogen gene encodes a trypsinogen protein having a substitution mutation at a position corresponding to R122 of human trypsinogen.

4. The transgenic animal of claim 3, wherein said heterologous gene comprises an arginine to histidine mutation at a position corresponding to amino acid 122 of human trypsinogen.

5. The transgenic animal of claim 1, wherein said animal is a rodent.

6. The transgenic animal of claim 5, wherein said rodent is a mouse.

7. The transgenic animal of claim 1, wherein said heterologous mutant trypsinogen gene encodes a trypsinogen protein having a substitution mutation at a position selected from the group consisting of positions corresponding to R122, A16 and N29 of human trypsinogen and combinations thereof.

8. A method of making a transgenic animal comprising the steps of:
a) introducing into an oocyte or embryonal cell of a non-human animal a polynucleotide sequence derived from said non-human animal that encodes a mutant trypsinogen protein, under conditions such that an embryonal target cell is produced;
b) transplanting said embryonal target cell, into a recipient female parent under conditions such that at least one offspring is produced; and
c) identifying said at least one offspring of said recipient female parent containing said polynucleotide sequence encoding said mutant trypsinogen protein, wherein said animal exhibits a phenotype selected from the group consisting of inflammatory destruction of exocrine pancreas, pre-neoplastic changes to pancreatic duct cells, periductal chronic inflammation, up-regulation of MMP-7, elevated fasting blood glucose levels, and high peaks in blood glucose after glucose administration and combinations thereof.

9. The method of claim 8, wherein said heterologous mutant trypsinogen protein has an altered autolysis site.

10. The method of claim 8, wherein said mutant trypsinogen protein has a substitution mutation at a position corresponding to R122 of human trypsinogen.

11. The method of claim 10, wherein said mutant trypsinogen protein has an arginine to histidine mutation at a position corresponding to amino acid 122 of human trypsinogen.

12. The method of claim 8, wherein said animal is a rodent.

13. The method of claim 12, wherein said rodent is a mouse.

14. The method of claim 8, wherein said mutant trypsinogen protein has a substitution mutation at a position selected from the group consisting of positions corresponding to R122, A16 and N29 of human trypsinogen and combinations thereof.

15. A method of identifying compounds, comprising:
a) providing at least one test compound and an animal whose genome comprises a heterologous mutant trypsinogen gene, wherein said animal exhibits a phenotype selected from the group consisting of inflammatory destruction of exocrine pancreas, pre-neoplastic changes to pancreatic duct cells, periductal chronic inflammation, up-regulation of MMP-7, elevated fasting blood glucose levels, and high peaks in blood glucose after glucose administration and combinations thereof
b) exposing said transgenic animal to said at least one test compound; and
c) detecting a change in at least one of said phenotypes in the presence of said test compound relative to the absence of said test compound.

16. The method of claim 15, wherein said test compound is a drug candidate.

17. The method of claim 15, wherein said heterologous mutant trypsinogen gene encodes a trypsinogen protein having an altered autolysis site.

18. The method of claim 15, wherein said heterologous mutant trypsinogen gene encodes a trypsinogen protein having a substitution mutation at a position corresponding to R122 of human trypsinogen.

19. The method of claim 18, wherein said heterologous gene comprises an arginine to histidine mutation at a position corresponding to amino acid 122 of human trypsinogen.

20. The method of claim 15, wherein said animal is a rodent.

21. The method of claim 20, wherein said rodent is a mouse.

22. The method of claim 15, wherein said heterologous mutant trypsinogen gene encodes a trypsinogen protein having a substitution mutation at a position selected from the group consisting of positions corresponding to R122, A16 and N29 of human trypsinogen and combinations thereof.

23. An isolated and purified nucleic acid sequence encoding a protein that is at least 98% identical to SEQ ID NO: 3 or 4.

24. The nucleic acid sequence of claim 23, wherein said sequence is operably linked to a heterologous promoter.

25. The nucleic acid sequence of claim 23, wherein said sequence is contained within a vector.

26. The nucleic acid sequence of claim 23, wherein said vector is within a host cell.

27. The nucleic acid of claim 23, wherein said protein has the amino acid sequence of SEQ ID NO: 3 or 4.


SEQUENCE LISTING (38 sequences)

SEQ ID NO: 1 - DNA, 765 bp, Mus musculus
atgagagcactcctgttcctggcccttgtgggagctgctgttgctttccctgtggatgatgatgacaagatcgttggaggatacacctgccgagagaattctgtcccctaccaggtgtccctgaactctggctaccacttctgtggaggttccctcatcaatgaccagtgggtggtgtctgcagctcactgctacaagtcccgcatccaagtgagactgggagaacacaacatcaatgtcctcgagggcaatgagcagtttgtcaattctgccaagatcatccgacaccccaattataactcgtggaccctgaacaatgacattatgctgatcaaactggcttcccctgtgaccctcaatgccagagtggccactgtggctctgcccagctcctgtgcacctgcaggcactcagtgtctcatctctggctggggcaacaccttgagctttggtgtgaacaacccagacctgctccagtgcctggatgccccattgctacctcaggctgactgtgaggcctcctatcctggaaagatcaccaataacatgatctgtgttggcttcctggagggaggcaaagattcctgccagggtgactctggtggtcctgtggtttgcaatggacagctccagggcattgtctcctggggctatggctgtgccctgaaggacaaccctggtgtgtacaccaaggtctgcaactatgtggactggattcaggacaccattgctgcaaacgattacaaggatgacgacgataagtaa

SEQ ID NO: 2 - DNA, 765 bp, Mus musculus
atgagagcactcctgttcctggcccttgtgggagctgctgttgctttccctgtggatgatgatgacaagatcgttggaggatacacctgccgagagaattctgtcccctaccaggtgtccctgaactctggctaccacttctgtggaggttccctcatcaatgaccagtgggtggtgtctgcagctcactgctacaagtcccgcatccaagtgagactgggagaacacaacatcaatgtcctcgagggcaatgagcagtttgtcaattctgccaagatcatccgacaccccaattataactcgtggaccctgaacaatgacattatgctgatcaaactggcttcccctgtgaccctcaatgcccacgtggccactgtggctctgcccagctcctgtgcacctgcaggcactcagtgtctcatctctggctggggcaacaccttgagctttggtgtgaacaacccagacctgctccagtgcctggatgccccattgctacctcaggctgactgtgaggcctcctatcctggaaagatcaccaataacatgatctgtgttggcttcctggagggaggcaaagattcctgccagggtgactctggtggtcctgtggtttgcaatggacagctccagggcattgtctcctggggctatggctgtgccctgaaggacaaccctggtgtgtacaccaaggtctgcaactatgtggactggattcaggacaccattgctgcaaacgattacaaggatgacgacgataagtaa

SEQ ID NO: 3 - protein, 254 aa, Mus musculus
MRALLFLALVGAAVAFPVDDDDKIVGGYTCRENSVPYQVSLNSGYHFCGGSLINDQWVVSAAHCYKSRIQVRLGEHNINVLEGNEQFVNSAKIIRHPNYNSWTLNNDIMLIKLASPVTLNARVATVALPSSCAPAGTQCLISGWGNTLSFGVNNPDLLQCLDAPLLPQADCEASYPGKITNNMICVGFLEGGKDSCQGDSGGPVVCNGQLQGIVSWGYGCALKDNPGVYTKVCNYVDWIQDTIAANDYKDDDDK

SEQ ID NO: 4 - protein, 254 aa, Mus musculus
MRALLFLALVGAAVAFPVDDDDKIVGGYTCRENSVPYQVSLNSGYHFCGGSLINDQWVVSAAHCYKSRIQVRLGEHNINVLEGNEQFVNSAKIIRHPNYNSWTLNNDIMLIKLASPVTLNAHVATVALPSSCAPAGTQCLISGWGNTLSFGVNNPDLLQCLDAPLLPQADCEASYPGKITNNMICVGFLEGGKDSCQGDSGGPVVCNGQLQGIVSWGYGCALKDNPGVYTKVCNYVDWIQDTIAANDYKDDDDK

SEQ ID NO: 5 - protein, 247 aa, Mus musculus
MKTLIFLAFLGAAVALPLDDDDDKIVGGYTCQRNALPYQVSLNSGYHFCGGSLINSQWVVSAAHCYKSRIQVRLGEHNIDALEGGEQFIDAAKIIRHPNYNANTYNNDIMLIKLKTAATLNSHVSTVALPRSCPSAGTRCLVSGWGNTLSSGTNYPSLLQCLDAPVLSDSSCTSSYPGKITSNMFCLGFLEGGKDSCQGDSGGPVVCNGQLQGVVSWGYGCAQRGKPGVYTKVCKYVNWIQQTIAAN

SEQ ID NO: 6 - protein, 245 aa, Mus musculus
MSALLFLALVGAAVAFPVDDDDKIVGGYTCRENSVPYQVSLNSGYHFCGGLINDQWVVSAAHCYKTRIQVRLGEHNINVLEGNEQFIDAAKIIKHPNFNRKTLNNDIMLIKLSSPVTLNAHVATVALPSSCAPAGTQCLISGWGNTLSFGVSEPDLLQCLDAPLLPQADCEASYPGKITGNMVCAGFLEGGKDSCQGDSGGPVVCNGELQGIVSWGYGCALPDNPGVYTKVCNYVDWIQDTIAAN

SEQ ID NO: 7 - protein, 246 aa, Mus musculus
MSALLILALVGAAVAFPVDDDDKIVGGYTCRESSVPYQVSLNAGYHFCGGSLINDQWVVSAAHCYKYRIQVRLGEHNINVLEGNEQFVDSAKIIRHPNYNSWTLDNDIMLIKLASPVTLNAHVASVPLPSSCAPAGTQCLISGWGNTLSNGVNNPDLLQCVDAPVLPQADCEASYPGDITNNMICVGFLEGGKDSCQGDSGGPVVCNGELQGIVSWGYGCAQPDAPGVYTKVCNYVDWIQNTIADN

SEQ ID NO: 8 - protein, 246 aa, Mus musculus
MNALLILALVGAAVAFPVDDDDKIVGGYTCQENSVPYQVSLNSGYHFCGGSLINDQWVVSAAHCYKTRIQVRLGEHNINVLEGNEQFVNAAKIIKHPNFNRKTLNNDIMLLKLSSPVTLNAHVATVALPSSCAPAGTQCLISGWGNTLSFGVSEPDLLQCLDAPLLPQADCEASYPGKITGNMVCAGFLEGGKDSCQGDSGGPVVCNRELQGIVSWGYGCALPDNPGVYTKVCNYVDWIQDTIAAN

SEQ ID NO: 9 - protein, 246 aa, Mus musculus
MRALLFLALVGAAVAFPVDDDDKIVGGYTCRENSVPYQVSLNSGYHFCGGSLINDQWVVSAAHCYKSRIQVRLGEHNINVLEGNEQFVNSAKIIKHPNFNSRTLNNDIMLIKLASPVTLNAHVATVALPSSCAPAGTQCLISGWGNTLSFGVNNPDLLQCLDAPLLPQADCEASYPGKITNNMICVGFLEGGKDSCQGDSGGPVVCNGQLQGIVSWGYGCALKDNPGVYTKVCNYVDWIQNTIAAN

SEQ ID NO: 10 - protein, 246 aa, Mus musculus
MNSLLFLALVGAAVAFPVDDDDKIVGGYTCRENSIPYQVSLNSGYHFCGGSLINDQWVVSAAHCYKTRIQVRLGEHNINVLEGNEQFVNSAKIIKHPNFNSRTLNNDIMLIKLASPVTLNAHVATVALPSSCAPAGTQCLISGWGNTLSFGVNNPDLLQCLDAPLLPQADCEASYPGKITNNMICVGFLEGGKDSCQGDSGGPVVCNGQLQGIVSWGYGCALKDNPGVYTKVCNYVDWIQDTIAAN

SEQ ID NO: 11 - DNA, 29 bp, Artificial Sequence
  Synthetic
cgcggatccatgagagcactcctgttcct

SEQ ID NO: 12 - DNA, 28 bp, Artificial Sequence
  Synthetic
cggggtaccttagtttgcagcaatggtg

SEQ ID NO: 13 - DNA, 25 bp, Artificial Sequence
  Synthetic
cgcgagctcaagcttatcgtatgaa

SEQ ID NO: 14 - DNA, 24 bp, Artificial Sequence
  Synthetic
tggccacgtgggcattgagggtca

SEQ ID NO: 15 - DNA, 23 bp, Artificial Sequence
  Synthetic
aatgcccacgtggccactgtggc

SEQ ID NO: 16 - DNA, 30 bp, Artificial Sequence
  Synthetic
ctagctagcggtaccttagtttgcagcaat

SEQ ID NO: 17 - DNA, 38 bp, Artificial Sequence
  Synthetic
cgcggatccaccttctgccaccatgagagcactcctgt

SEQ ID NO: 18 - DNA, 30 bp, Artificial Sequence
  Synthetic
gtcatccttgtaatcgtttgcagcaatggt

SEQ ID NO: 19 - DNA, 30 bp, Artificial Sequence
  Synthetic
ctagctagcttacttatcgtcgtcatcctt

SEQ ID NO: 20 - DNA, 24 bp, Artificial Sequence
  Synthetic
atggtgagcaagggcgaggagctg

SEQ ID NO: 21 - DNA, 24 bp, Artificial Sequence
  Synthetic
cagctcgtccatgccgagagtgat

SEQ ID NO: 22 - DNA, 24 bp, Artificial Sequence
  Synthetic
gccatgcccgaaggctacgtccag

SEQ ID NO: 23 - DNA, 744 bp, Mus musculus
atgaagaccttaatcttccttgccttccttggagctgctgttgctctccccctggatgatgatgatgacaaaattgttggaggctacacctgccagaggaatgctctcccctaccaggtgtctctgaacagcggctaccatttttgtggaggctcactcatcaattcccagtgggttgtttcagccgctcactgctacaaatcccgaattcaggtgcgcctgggagaacacaacattgatgcccttgagggtggtgagcaattcattgatgcagctaaaatcatccgccaccccaactataatgcaaacacctataacaatgacatcatgttgattaagctgaagacagctgccaccctcaactctcacgtgtccactgtcgctctgccaagatcttgtccatcagctggtactaggtgtcttgtgtctggctggggcaacaccctgagctctggcaccaactacccttcactccttcagtgtcttgatgctcctgtcctctctgacagttcttgcacaagttcttacccaggcaagatcactagcaacatgttctgtctgggcttcctggagggcggaaaggactcctgccagggtgactctggtggccccgtagtctgcaatggacaactccagggtgttgtttcctggggttatggctgtgctcagagaggaaaacctggtgtctacaccaaggtgtgcaagtatgtgaactggattcagcagaccatcgctgccaactaa

SEQ ID NO: 24 - DNA, 741 bp, Mus musculus
atgagtgcactcctgttcctggcccttgtgggagctgctgttgctttccctgtggatgatgatgacaagatcgttggaggatacacctgccgagagaattctgttccctaccaggtgtccctgaactctggctaccacttctgtggaggttccctcatcaatgaccagtgggtggtgtctgcagctcattgctacaagacccgcatccaagtgagattgggggagcacaatatcaatgtcctggagggcaatgagcagtttatcgatgctgccaagatcatcaagcaccccaacttcaataggaagaccctgaacaatgacatcatgttgatcaagctctcatcccctgtgaccctcaatgcccacgtggccactgtggctctgcccagctcctgtgcacctgctggcactcagtgtctcatctctggctggggcaacactcttagctttggtgtcagtgaaccagacctgctccagtgcctggatgccccactgctgcctcaggctgactgtgaggcttcctatcctgggaagatcactggtaacatggtctgtgctggcttcctagagggaggcaaggattcctgccagggtgactctggtggccctgtggtctgcaatggagagctccagggcattgtctcttggggctatggctgtgccctgccagacaatcctggtgtgtacaccaaggtctgcaactatgtggattggattcaggacacaattgctgccaactag

SEQ ID NO: 25 - DNA, 741 bp, Mus musculus
atgaatgcactcctgatcctggcccttgtgggagctgctgttgccttccccgtggatgatgatgacaagatcgttggaggatacacctgccaagagaattctgtcccctaccaggtgtccctgaactctggctaccacttctgtggaggctccctcatcaatgaccagtgggtggtgtctgcagctcactgctacaaaacccgcatccaagtgagattgggggagcacaacatcaacgtcctggagggcaatgagcagtttgtcaatgctgccaagatcatcaagcaccccaacttcaataggaagacactgaacaacgacattatgttgctcaagctctcttcccctgtgaccctcaatgcccacgtggccactgtggctctgcccagctcctgtgcacctgctggcactcagtgtctcatctctggctggggcaacactcttagctttggtgtcagtgaaccagacctgctccagtgcctggatgccccactgctgcctcaggctgactgtgaagcctcctatcctgggaagatcactggtaacatggtctgtgctggcttcctagagggaggcaaggattcctgccagggtgactccggtggccctgtggtctgcaatagagaactccagggtatagtctcctggggctatggctgtgccctgccagacaatcctggtgtgtacaccaaggtctgcaactatgtggactggattcaggacacaattgctgccaactag

SEQ ID NO: 26 - DNA, 741 bp, Mus musculus
atgagagcactcctgttcctggcccttgtgggagctgctgttgctttccctgtggatgatgatgacaagatcgttggaggatacacctgccgagagaattctgtcccctaccaggtgtccctgaactcaggctaccacttctgtggaggttccctcatcaatgaccagtgggtggtgtctgcagctcactgctacaagtcccgcatccaagtgagactgggagagcacaacatcaatgtcctcgagggcaatgagcagtttgtcaattctgccaagatcatcaagcatcccaacttcaatagcaggaccctgaacaacgacatcatgctgatcaaactggcttcccctgtgaccctcaatgcccacgtggccactgtggctctgcccagctcctgtgcacctgcaggcactcagtgtctcatctctggctggggcaacaccttgagctttggtgtgaacaacccagacctgctccagtgcctggatgccccactgctgcctcaggctgactgtgaggcctcctatcctggaaagatcaccaataacatgatctgtgttggcttcctagagggaggcaaagattcctgccagggtgactctggtggccctgtggtctgcaatggacagctccagggcattgtctcctggggctatggctgtgccctgaaggacaaccctggtgtgtacaccaaggtctgcaactatgtggactggattcaaaacaccattgctgcaaactaa

SEQ ID NO: 27 - DNA, 741 bp, Mus musculus
atgagtgcacttctgatcctagcccttgtgggagctgctgttgctttccctgtggatgatgatgacaagattgttggaggatacacctgccgagagagttctgtcccctatcaggtgtccctaaatgctggctaccacttctgtggaggttccctcatcaatgaccagtgggtggtgtctgcagctcactgctacaaataccgcatccaagtgagactgggagagcacaacatcaatgtcctggagggcaatgagcagtttgttgattctgccaagatcatccggcaccccaattataattcatggaccctggacaatgacatcatgctgatcaaactggcttcccctgtgaccctcaatgcccacgtggcctctgtacctctgcccagctcctgtgcacctgcaggcactcagtgcctcatctctggctggggcaacaccctcagcaatggtgtgaacaacccagacctgctccagtgtgttgatgccccagtgctgcctcaggctgactgtgaggcctcctaccctggggacatcaccaacaacatgatctgtgttggcttcctggagggaggcaaagattcctgccagggtgactctggtggccctgtggtctgcaatggagagctgcagggcattgtctcctggggctatggctgtgcccagccagatgctcctggtgtatacaccaaggtctgcaactacgtggactggattcagaacacaattgctgacaactag

SEQ ID NO: 28 - DNA, 741 bp, Mus musculus
atgaattcactcctgttcctggcccttgtgggagctgctgttgctttccctgtggatgatgatgacaagatcgttggaggatacacctgccgagagaattctatcccctaccaggtgtctctgaactctggctaccacttctgtggaggttccctcatcaatgaccagtgggtggtgtctgcagctcattgctacaaaacccgcatccaagtgagactgggagagcacaacatcaatgtcctcgagggcaatgagcagtttgtcaattctgccaagatcatcaagcatcccaacttcaatagcaggaccctgaacaacgacatcatgctgatcaaactggcttcccctgtgaccctcaatgcccacgtggccactgtggctctgcccagctcctgtgcacctgcaggcactcagtgtctcatctctggctggggcaacaccttgagctttggtgtgaacaacccagacctgcttcaatgcctggatgccccattgctacctcaggctgactgtgaggcctcttatcctggaaagatcaccaataacatgatctgtgttggcttcctggagggaggcaaagattcctgccagggtgactctggtggtcctgtggtttgcaatggacagctccagggcattgtctcctggggctatggctgtgccctgaaggacaaccctggtgtgtacaccaaggtctgcaactatgtggactggattcaggacaccattgccgcaaactaa

SEQ ID NO: 29 - DNA, 741 bp, Mus musculus; identical to SEQ ID NO: 27

SEQ ID NO: 30 - DNA, 741 bp, Mus musculus; identical to SEQ ID NO: 28

SEQ ID NO: 31 - DNA, 741 bp, Mus musculus
atgagtacactcctgttcctggcccttgtgggagcagctgttgctttccctgtggatgatgatgacaagatagttggaggatacacctgccgagagaattctgttccctaccaggtgtccctgaactctggctaccacttctgtggaggttccctcatcaatgaccagtgggtggtgtctgcagctcactgctacaagtcccgcatccaagtgagactgggagagcacaacatcaacgtcctggagggcaatgagcagtttatcgatgctgccaacatcatcaagcatcctaaattcaaaaagaagactctggacaatgacatcatgctgatcaagctgtcttctcctgtgaccctcaatgcccacgtggccactgtggctctgcccagctcctgtgcagctgctggcactcagtgcctcatctctggctggggcaacaccttgagctctggcgtgaacaacccagacctgctccagtgcctggatgctccactgctgcctcaggctgactgtgaggcctcctatcctggaaagatcaccaagaacatgatctgtgttggcttcctggagggaggcaaagattcctgccagggtgactctggtggccctgtggtctgcaatggacagctccagggcattgtttcttggggctatggctgtgcccagaaggacaaccctggtgtgtacaccaaggtctgcaactatgtggactggattcaaaacaccattgctgcaaactaa

SEQ ID NO: 32 - DNA, 738 bp, Mus musculus
atgagtgcactcctcttcctggcccttgtgggagctgctgttgctttccctgtggatgatgataagatcgttggaggatacacctgccgagagaattctgttccctaccaggtgtccctgaactctggctaccacttctgtggaggttccctcatcaatgaccagtgggtggtgtctgcagctcattgctacaaaacccgcatccaagtgagattgggggagcacaacatcaaagtcctggagggcaatgagcagtttgtcaatgctgccaagatcatcaagcaccccaacttcaataggaagaccctcaataacgacatcatgttgatcaagctctcttcccctgtgaccctcaatgcccacgtggccactgtggctctgcccagctcctgtgcgccagctggcactcagtgtctcatctctggctggggcaacactcttagctttggtgtcagtgaaccagacctgctccagtgcctggatgccccactgctgcctcaggctgactgtgaagcctcctatcctgggaagatcactggtaacatggtctgtgctggcttcctagagggaggcaaggattcctgccagggtgactctggtggccctgtggtctgcaatggagagctccagggcattgtctcttggggctatggctgtgccctggcagacaatcctggtgtgtacaccaaggtctgcaactatgtggattggattcaggacacaattgctgccaactag

SEQ ID NO: 33 - DNA, 741 bp, Mus musculus
atgaatgcattcctgatcctggccctggtgggagctgctgttgccttccccgtggatgatgatgacaagatcgttggaggatacacctgccaagagaattctgttccctaccaggtgtccctgaactctggctaccacttctgtggaggctccctcatcaatgaccagtgggtggtgtctgcagctcactgctacaaaacccgcatccaagtgagattgggggagcacaacatcaacgtcctggagggcaatgagcagtttgtcaatgctgccaagatcatcaagcaccccaacttcaataggaagacactgaacaacgacatcatgttgatcaagctctcttcccctgtgaccctcaatgcccacgtggccactgtggctctgcccagctcctgtgcacctgctggcactcagtgtctcatctctggctggggcaacactcttagctttggtgtcagtgaaccagacctgctccagtgcctggatgccccactgctgcctcaggctgactgtgaagcctcctatcctgggaagatcactggtaacatggtctgtgctggcttcctagagggaggcaaggattcctgccagggtgactccggtggccctgtggtctgcaatggagagctccagggtatagtctcctggggctatggctgtgccctgccagacaatcctggtgtgtacaccaaggtctgcaactatgtggactggattcaggacacaattgctgccaactag

SEQ ID NO: 34 - protein, 246 aa, Mus musculus; identical to SEQ ID NO: 7

SEQ ID NO: 35 - protein, 246 aa, Mus musculus; identical to SEQ ID NO: 10

SEQ ID NO: 36 - protein, 246 aa, Mus musculus
MSTLLFLALVGAAVAFPVDDDDKIVGGYTCRENSVPYQVSLNSGYHFCGGSLINDQWVVSAAHCYKSRIQVRLGEHNINVLEGNEQFIDAANIIKHPKFKKKTLDNDIMLIKLSSPVTLNAHVATVALPSSCAAAGTQCLISGWGNTLSSGVNNPDLLQCLDAPLLPQADCEASYPGKITKNMICVGFLEGGKDSCQGDSGGPVVCNGQLQGIVSWGYGCAQKDNPGVYTKVCNYVDWIQNTIAAN

SEQ ID NO: 37 - protein, 245 aa, Mus musculus
MSALLFLALVGAAVAFPVDDDKIVGGYTCRENSVPYQVSLNSGYHFCGGSLINDQWVVSAAHCYKTRIQVRLGEHNIKVLEGNEQFVNAAKIIKHPNFNRKTLNNDIMLIKLSSPVTLNAHVATVALPSSCAPAGTQCLISGWGNTLSFGVSEPDLLQCLDAPLLPQADCEASYPGKITGNMVCAGFLEGGKDSCQGDSGGPVVCNGELQGIVSWGYGCALADNPGVYTKVCNYVDWIQDTIAAN

SEQ ID NO: 38 - protein, 246 aa, Mus musculus
MNAFLILALVGAAVAFPVDDDDKIVGGYTCQENSVPYQVSLNSGYHFCGGSLINDQWVVSAAHCYKTRIQVRLGEHNINVLEGNEQFVNAAKIIKHPNFNRKTLNNDIMLIKLSSPVTLNAHVATVALPSSCAPAGTQCLISGWGNTLSFGVSEPDLLQCLDAPLLPQADCEASYPGKITGNMVCAGFLEGGKDSCQGDSGGPVVCNGELQGIVSWGYGCALPDNPGVYTKVCNYVDWIQDTIAAN